(19) United States
(12) Patent Application Publication
(10) Pub. No.: US 2003/0135105 A1
(43) Pub. Date: Jul. 17, 2003

(54) Alignment of multiple MR images using navigator signals

(76) Inventors: Clifford R. Jack, Rochester, MN (US); Armando Manduca, Rochester, MN (US); Edward Brian Welch, Rochester, MN (US); Roger C. Grimm, Rochester, MN (US)

Correspondence Address: Barry E. Sammons, Quarles & Brady, LLP, 411 East Wisconsin Avenue, West Bend, WI, 53202, US

(21) Appl. No.: 10/326,734

(22) Filed: Dec. 19, 2002

Related U.S. Application Data
(63) Continuation-in-part of application No. PCT/US01/12355, filed on Apr. 16, 2001.
(60) Provisional application No. 60/199,854, filed on Apr. 26, 2000.
(60) Provisional application No. 60/210,929, filed on Jun. 12, 2000.

(51) Int. Cl. A61B 5/05

(52) U.S. Cl. 600/410

(57) ABSTRACT

A series of MR examinations of a patient are performed and the acquired images are aligned with each other so that small anatomic changes can be detected when images are compared. Alignment is achieved by acquiring navigator signals during each examination which are analyzed to measure patient misalignment from one examination to the next. The rotational and translational misalignment information is used to either prospectively or retrospectively align the MR images.

### RELATED APPLICATIONS

This application is a continuation-in-part of international application PCT/US01/12355 filed in the United States Patent and Trademark Office on Apr. 16, 2001 which claims benefit of provisional application Serial Nos. 60/199,854 and 60/210,929 filed in the United States Patent and Trademark Office on Apr. 26, 2000 and Jun. 12, 2000.

### STATEMENT REGARDING FEDERALLY SPONSORED RESEARCH

This invention was made with government support under Grant No. AG19142 awarded by the National Institute of Health. The United States Government has certain rights in this invention.

### BACKGROUND OF THE INVENTION

The field of the invention is nuclear magnetic resonance imaging methods and systems. More particularly, the invention relates to the alignment of NMR images acquired from a subject during a series of examinations.

There are a number of clinical situations in which magnetic resonance images (“MRI”) are acquired at different times and then compared to each other. For example, as a routine part of clinical management, patients with brain tumors are imaged serially over the course of treatment to assess the progression of the disease. In order to do this, the radiologist must align, or register, successive images precisely and visually compare tumor size. If the tumor changes grossly in size, such interpretation is not difficult despite image misalignment. However, frequently this change in tumor size can be very small and the changes very subtle from one image to the next. Absent a method for precisely aligning the subject in the MRI system from one examination to the next, the radiologist's interpretation is often inconclusive.

Numerous devices and methods are known for aligning a patient in a medical imaging system with respect to its coordinate system. Immobilization apparatus such as that disclosed in U.S. Pat. No. 5,800,353 may be employed to align the subject in the same location with respect to the MRI system imaging coordinate systems from one examination to the next. Such devices require time to set up and use, and the registration of successive images is not accurate enough for many clinical situations.

Fiducial marks or fiducial implants may also be placed on the subject as described in U.S. Pat. Nos. 6,226,418; 5,299,253; 5,901,199 and 5,531,520 and used to align the patient at successive examinations or to register successive images. In some cases the marks may be employed to align the patient using external devices such as lasers or video cameras and in some cases the resulting bright objects produced in the acquired images are employed to align the images. The use of fiducials is not desirable when the examinations occur over a long period of time because they can wear off or shift location on the subject.

Another approach is to register the successive images using brute force least-squares estimation, iterative least-squares estimation and cross correlation methods as described for example in U.S. Pat. Nos. 5,850,486 and 5,295,200. These methods are very computer intensive because they rely on repetitive, complex calculations involving all the image pixel magnitudes to align the successive images. For best performance, this image registration method also assumes that the images are the same over time which, of course, is usually not true in a clinical setting.

U.S. Pat. No. 4,937,526 describes a method for reducing motion artifacts in NMR images in which the NMR data set used to reconstruct the image is corrected after its acquisition using information acquired concurrently in NMR “navigator” signals. The navigator signals are produced by pulse sequences which are interleaved with the imaging pulse sequences and which are characterized by the absence of phase encoding. The navigator signal is thus a projection along an axis defined by the readout gradient which is fixed in direction throughout the scan. As a result, the navigator signals detect spin motion only along the direction of this readout gradient. A second navigator pulse sequence with an orthogonal readout gradient can also be interleaved throughout the scan, but this further lengthens the scan time and is seldom done. In addition, even when two “orthogonal” navigator signals are acquired during the scan, they do not provide the information required to correct for in-plane rotation of the subject. Such rotational motion is particularly troublesome when imaging certain subjects such as the human heart, or when performing brain function MRI.

The difficulty in correcting for rotational motion has been solved as described in U.S. Pat. No. 5,539,312. Navigator signals are acquired using a unique pulse sequence which samples two-dimensional k-space in a circular trajectory. These “orbital” navigator signals are used to correct NMR image data for rotation and translation in a single two-dimensional plane. To obtain sufficient information to correct for all possible rotations and translations, the orbital navigator pulse sequence must be performed three times.

### SUMMARY OF THE INVENTION

The present invention is a method for acquiring magnetic resonance images during a plurality of separate examinations and aligning those images so that they can be compared. In addition to acquiring MR image data during each examination, navigator signal data is acquired and stored. Misalignment of the subject from one examination to the next is determined by analyzing the corresponding navigator signal data and either prospectively adjusting the imaging pulse sequence, or retrospectively producing corrections to image signal magnitude and phase which effectively align the images.

In a preferred embodiment of the invention the images are aligned to account for both subject translation and subject rotation between examinations. This is achieved by performing a spherical navigator pulse sequence including the application of three orthogonal magnetic field gradients during the readout of its spherical navigator NMR signal such that the spherical navigator NMR signal samples a substantially spherical surface in three-dimensional k-space.

In a preferred embodiment of the invention navigator signal data is acquired during subsequent examinations and compared with the reference navigator signal data to measure patient misalignment. This information is then employed prospectively to adjust the imaging pulse sequence such that the MRI system imaging coordinate system is rotated and/or translated an offsetting amount. The subsequently acquired image of the patient is thus aligned with the reference image.

In another embodiment of the invention the navigator signal data acquired with a reference image of the patient is compared with the navigator signal data acquired with a subsequent image of the patient and the subsequent image is retrospectively rotated and translated to align the patient in the two images. The two images can then be compared by the clinician to see any changes in the patient that may have occurred.

The foregoing and other objects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims herein for interpreting the scope of the invention.

### BRIEF DESCRIPTION OF THE DRAWINGS

FIG. 1 is a block diagram of an NMR system which has been modified to practice the present invention;

FIG. 2 is a flow chart of the preferred MRI examination method which employs the present invention;

FIG. 3 is a graphic representation of a preferred embodiment of the spherical navigator pulse sequence of the present invention;

FIG. 4 is a graphic representation of the spherical sampling of k-space performed by the pulse sequence of FIG. 3;

FIG. 5 is a flow chart of the method used to align image data using navigator signals acquired with the pulse sequence of FIG. 3;

FIG. 6 is a graph showing the reliability of the motion measurement as a function of number of navigator signal samples; and

FIGS. 7a-c are pictoral representations of the sampled spherical surface and resulting texture maps.

### GENERAL DESCRIPTION OF THE INVENTION

A spherical navigator (SNAV) dataset is formed by acquiring data points which describe a spherical 3D shell in k-space. The SNAV dataset is acquired during each MRI examination of a patient, and the patient images are aligned by comparing the current SNAV dataset to its predecessor in time. Rotations of the patient are encoded in the magnitude of the SNAV signal and translations are encoded in the phase of the signal. The single SNAV dataset contains information about rotation and translation of the object in all three dimensions.

The degrees of rotational freedom may be expressed as successive rotations about the x, y, z axes in that order. Other ways of expressing 3D rotations exist, but this is a convenient representation and is suitable when the rotation angles are small. In this representation, a point (x, y, z) is mapped from a point (x′, y′, z′) by rotations θₓ, θ_{y}, θ_{z} and translations x₀, y₀, z₀ by:

$$[ \begin{matrix}x \\ y \\ z\end{matrix} ] = M [ \begin{matrix}{x}^{′} \\ {y}^{′} \\ {z}^{′}\end{matrix} ] + [ \begin{matrix}{x}_{0} \\ {y}_{0} \\ {z}_{0}\end{matrix} ]$$

$$where M = [ \begin{matrix}{c}_{y} {c}_{z} & {s}_{x} {s}_{y} {c}_{z} - {c}_{x} {s}_{z} & {c}_{x} {s}_{y} {c}_{z} + {s}_{x} {s}_{z} \\ {c}_{y} {s}_{z} & {s}_{x} {s}_{y} {s}_{z} - {c}_{x} {c}_{z} & {c}_{x} {s}_{y} {s}_{z} - {s}_{x} {c}_{z} \\ - {s}_{y} & {s}_{x} {c}_{y} & {c}_{x} {c}_{y}\end{matrix} ]$$

and where cₓ=cos(θₓ), s_{y}=sin (θ_{y}), etc.

In k-space, the same rotation matrix applies, but the translations become phase terms. A signal S′ measured at the new location (kₓ, k_{y}, k_{z}) or (kρ, θ′, φ′ in polar coordinates) by:

$$\begin{matrix}{S}^{′} ( {k}_{x} , {k}_{y} , {k}_{z} ) = {S}^{′} ( {k}_{p} , θ , φ ) = S ( {k}_{p} , {θ}^{′} , {φ}^{′} ) {ⅇ}^{ⅈ2 π ( {k}_{x} {x}_{0} + {k}_{y} {y}_{0} + {k}_{z} {z}_{0} )} \\ = S ( {k}_{ρ} , {θ}^{′} , {φ}^{′} ) {ⅇ}^{ⅈ2π {k}_{ρ}^{( {x}_{0} cos θcos φ + {y}_{0} sin θ cos φ + {z}_{0} sin φ )}} .\end{matrix} \qquad ( 1 )$$

There are no simple direct formulas for θ and φ in terms of θ′ and φ′, but they can be deduced from (kₓ, k_{y}, k_{z}). Notice that k_{ρ} does not change. Rotations of an object in space correspond to rotations in k-space, in which points simply rotate on a spherical surface and their magnitude values do not change. Translations simply add phase shifts to points in k-space, and thus do not affect the magnitude values. Off-center rotations are equivalent to an on-center rotation plus an apparent translation of the coordinate frame. Also, one should note that in 3D any combination of rotations is equivalent to a single rotation about some axis.

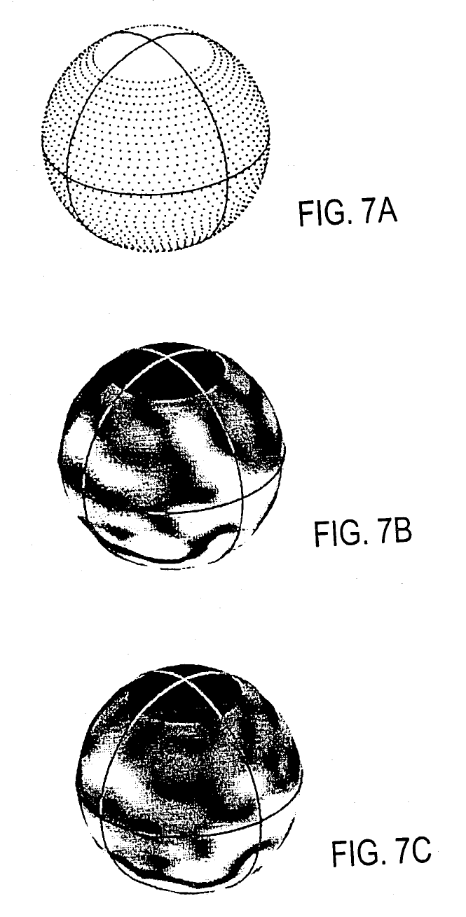

In order to detect a change in rotation of a given SNAV dataset (SNAVₙ) with respect to its reference, or baseline (SNAV₀), SNAVₙ is rotated about the origin of 3D k-space, and the magnitude values on the surface of SNAVₙ are compared with those on the surface of SNAV₀ at each new rotational position. The magnitude data on a spherical surface in k-space at an appropriate radius has features. This “intensity texture” of the SNAVs simply rotates with arbitrary 3D rotations, so the patterns before and after a rotation can be matched, or registered, and the rotation parameters that yield the best registration are recorded. This is a registration problem, analogous to rotating the earth's surface in an arbitrary way and deducing the rotation parameters by “lining up” the mountain ranges and valleys. This registration process is straightforward provided that there are sufficient features on the spherical surface and that it is sampled densely enough. FIGS. 7a-c illustrate (from left to right) the bi-hemispheric K-space sampling scheme that is employed in the preferred method; a texture map derived from the SNAV₀ data in base-line position; and, a texture map of the same object after a rotation. The great circles are intended to aid visualization of rotation of the texture features with respect to the constant position of the great circles.

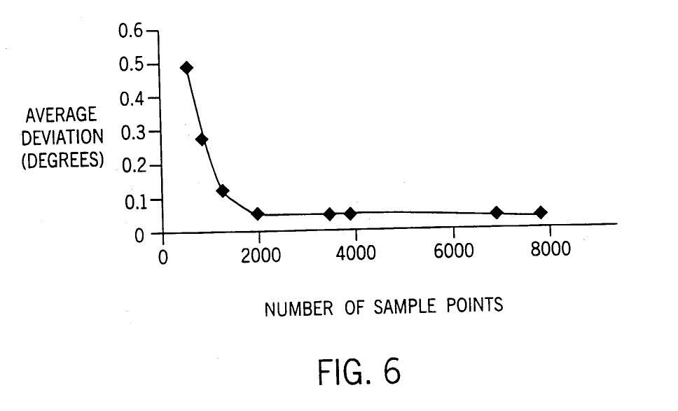

Experiments have been conducted to determine the accuracy of the motion measurements and the minimum number of sample points required to obtain this accuracy. Spherical k-space surfaces were sampled substantially uniformly at different densities and used to measure rotation of a phantom. FIG. 6 is a graph which shows the deviation of the measured phantom rotation using progressively smaller numbers of k-space samples. It was discovered that the measurements do not deviate significantly until less than 1000 samples are acquired. The optimal number of samples of the k-space spherical surface is in the range of 1000 to 2000 samples. This is significant in that this number of samples can be obtained during a single pulse sequence. The precision of the method is within +0.1 for all axes when 1952 samples of the k-space surface are acquired.

Experiments have also been conducted to determine the dynamic range of the SNAV measurements and their sensitivity. These measurements indicate that movements up to ±5° of rotation and up to ±5 mm of translation can be measured, and that the measurements have submillimeter and subdegree accuracy. The accuracy of motion detection is substantially equivalent to prior navigator signal measurement methods.

Another variable in the SNAV method is the radius k_{ρ} of the spherical surface. The SNR of acquired SNAV signals is proportional to k_{ρ}^{-1/2} with the result that increased spherical radius decreases the SNAV signal-to-noise ratio. However, a larger radius k_{ρ} increases the spatial detail in the sampled subject resulting in a more accurate registration of the acquired SNAVₙ and the reference SNAV₀. The radius k_{ρ} is limited by the maximum gradient slew rates on the MRI system. At maximum gradient slew rates, k_{ρ} is inversely proportional to the number of turns on the spherical surface. The radius kp used in the preferred embodiment is 9.5.

### DESCRIPTION OF THE PREFERRED EMBODIMENT

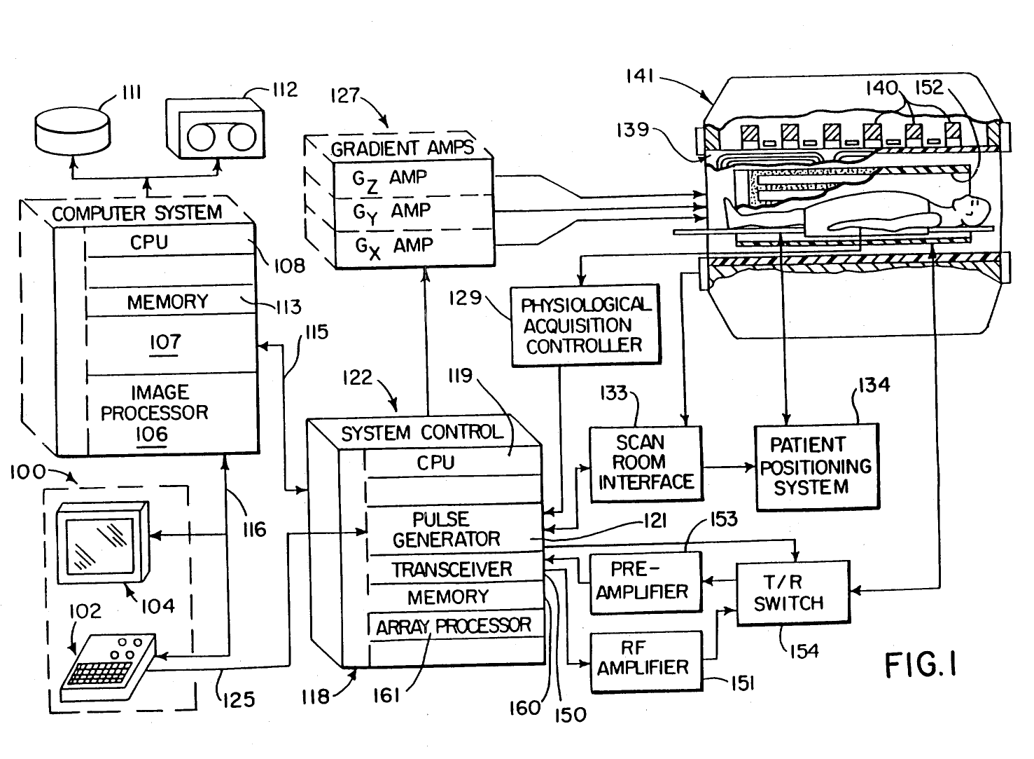

Referring first to FIG. 1, there is shown the major components of a preferred NMR system which incorporates the present invention and which is sold by the General Electric Company under the trademark “SIGNA”. The operation of the system is controlled from an operator console 100 which includes a console processor 101 that scans a keyboard 102 and receives inputs from a human operator through a control panel 103 and a plasma display/touch screen 104. The console processor 101 communicates through a communications link 116 with an applications interface module 117 in a separate computer system 107. Through the keyboard 102 and controls 103, an operator controls the production and display of images by an image processor 106 in the computer system 107, which connects directly to a video display 118 on the console 100 through a video cable 105.

The computer system 107 includes a number of modules which communicate with each other through a backplane. In addition to the application interface 117 and the image processor 106, these include a CPU module 108 that controls the backplane, and an SCSI interface module 109 that connects the computer system 107 through a bus 110 to a set of peripheral devices, including disk storage 111 and tape drive 112. The computer system 107 also includes a memory module 113, known in the art as a frame buffer for storing image data arrays, and a serial interface module 114 that links the computer system 107 through a high speed serial link 115 to a system interface module 120 located in a separate system control cabinet 122.

The system control 122 includes a series of modules which are connected together by a common backplane 118. The backplane 118 is comprised of a number of bus structures, including a bus structure which is controlled by a CPU module 119. The serial interface module 120 connects this backplane 118 to the high speed serial link 115, and pulse generator module 121 connects the backplane 118 to the operator console 100 through a serial link 125. It is through this link 125 that the system control 122 receives commands from the operator which indicate the scan sequence that is to be performed.

The pulse generator module 121 operates the system components to carry out the desired scan sequence. It produces data which indicates the timing, strength and shape of the RF pulses which are to be produced, and the timing of and length of the data acquisition window. The pulse generator module 121 also connects through serial link 126 to a set of gradient amplifiers 127, and it conveys data thereto which indicates the timing and shape of the gradient pulses that are to be produced during the scan. The pulse generator module 121 also receives patient data through a serial link 128 from a physiological acquisition controller 129. The physiological acquisition control 129 can receive a signal from a number of different sensors connected to the patient. For example, it may receive ECG signals from electrodes or respiratory signals from a bellows and produce pulses for the pulse generator module 121 that synchronizes the scan with the patient's cardiac cycle or respiratory cycle. And finally, the pulse generator module 121 connects through a serial link 132 to scan room interface circuit 133 which receives signals at inputs 135 from various sensors associated with the position and condition of the patient and the magnet system. It is also through the scan room interface circuit 133 that a patient positioning system 134 receives commands which move the patient cradle and transport the patient to the desired position for the scan.

The gradient waveforms produced by the pulse generator module 121 are applied to a gradient amplifier system 127 comprised of Gₓ, G_{y} and G_{z} amplifiers 136, 137 and 138, respectively. Each amplifier 136, 137 and 138 is utilized to excite a corresponding gradient coil in an assembly generally designated 139. The gradient coil assembly 139 forms part of a magnet assembly 141 which includes a polarizing magnet 140 that produces either a 0.5 or a 1.5 Tesla polarizing field that extends horizontally through a bore 142. The gradient coils 139 encircle the bore 142, and when energized, they generate magnetic fields in the same direction as the main polarizing magnetic field, but with gradients Gₓ, G_{y} and G_{z} directed in the orthogonal x-, y- and z-axis directions of a Cartesian coordinate system. That is, if the magnetic field generated by the main magnet 140 is directed in the z direction and is termed B₀, and the total magnetic field in the z direction is referred to as B_{z}, then Gₓ=∂B_{z}/∂x, G_{y}=∂B_{z}/∂y and G_{z}=∂B_{z}/∂z, and the magnetic field at any point (x,y,z) in the bore of the magnet assembly 141 is given by B(x,y,z)=B₀+Gₓx+G_{y}y+G_{z}z. The gradient magnetic fields are utilized to encode spatial information into the NMR signals emanating from the patient being scanned.

Located within the bore 142 is a circular cylindrical whole-body RF coil 152. This coil 152 produces a circularly polarized RF field in response to RF pulses provided by a transceiver module 150 in the system control cabinet 122. These pulses are amplified by an RF amplifier 151 and coupled to the RF coil 152 by a transmit/receive switch 154 which forms an integral part of the RF coil assembly. Waveforms and control signals are provided by the pulse generator module 121 and utilized by the transceiver module 150 for RF carrier modulation and mode control. The resulting NMR signals radiated by the excited nuclei in the patient may be sensed by the same RF coil 152 and coupled through the transmit/receive switch 154 to a preamplifier 153. The amplified NMR signals are demodulated, filtered, and digitized in the receiver section of the transceiver 150. The transmit/receive switch 154 is controlled by a signal from the pulse generator module 121 to electrically connect the RF amplifier 151 to the coil 152 during the transmit mode and to connect the preamplifier 153 during the receive mode. The transmit/receive switch 154 also enables a separate RF coil (for example, a head coil or surface coil) to be used in either the transmit or receive mode.

In addition to supporting the polarizing magnet 140 and the gradient coils 139 and RF coil 152, the main magnet assembly 141 also supports a set of shim coil 156 associated with the main magnet 140 and used to correct inhomogeneities in the polarizing magnet field. The main power supply 157 is utilized to bring the polarizing field produced by the superconductive main magnet 140 to the proper operating strength and is then removed.

The NMR signals picked up by the RF coil 152 are digitized by the transceiver module 150 and transferred to a memory module 160 which is also part of the system control 122. When the scan is completed and an entire array of data has been acquired in the memory modules 160, an array processor 161 operates to Fourier transform the data into an array of image data. This image data is conveyed through the serial link 115 to the computer system 107 where it is stored in the disk memory 111. In response to commands received from the operator console 100, this image data may be archived on the tape drive 112, or it may be further processed by the image processor 106 and conveyed to the operator console 100 and presented on the video display 118.

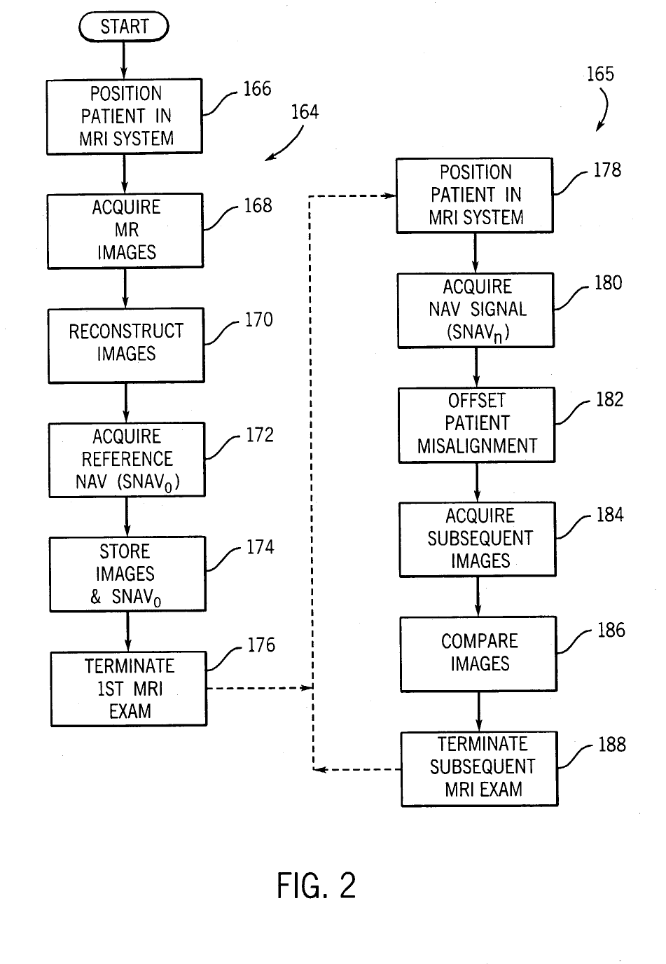

Referring particularly to FIG. 2, the present invention is a method for operating the MRI system of FIG. 1 to perform a series of MRI examinations of a patient over time and to automatically align the resulting images so that they can be compared for diagnostic purposes. As will be described in more detail below, a reference navigator signal (SNAV₀) is acquired during the initial patient MRI examination indicated generally at 164 and this reference navigator signal is used to align images acquired during subsequent MRI examinations indicated generally at 165. The MR images acquired from one examination to the next will typically have the same prescription, but these may be acquired using any of the known imaging pulse sequences, such as spin echo, gradient echo, fast spin echo, fast gradient echo, echo planer imaging (EPI), etc. In most clinical applications the objective is to repeat the same MRI examination over time and compare the images to determine what changes, if any, have occurred. Monitoring the stages of a malignant tumor during treatment is a typical clinical application of the present invention.

Referring particularly to FIG. 2, during the initial examination the patient is positioned in the MRI system as indicated by process block 166. Patient positioning and alignment devices may be employed during this step, although it may be as simple as placing the patient on the table and moving the table to a selected location. The prescribed imaging pulse sequence is then selected and one or more MR images are acquired as indicated at process block 168 and reconstructed as indicated at process block 170.

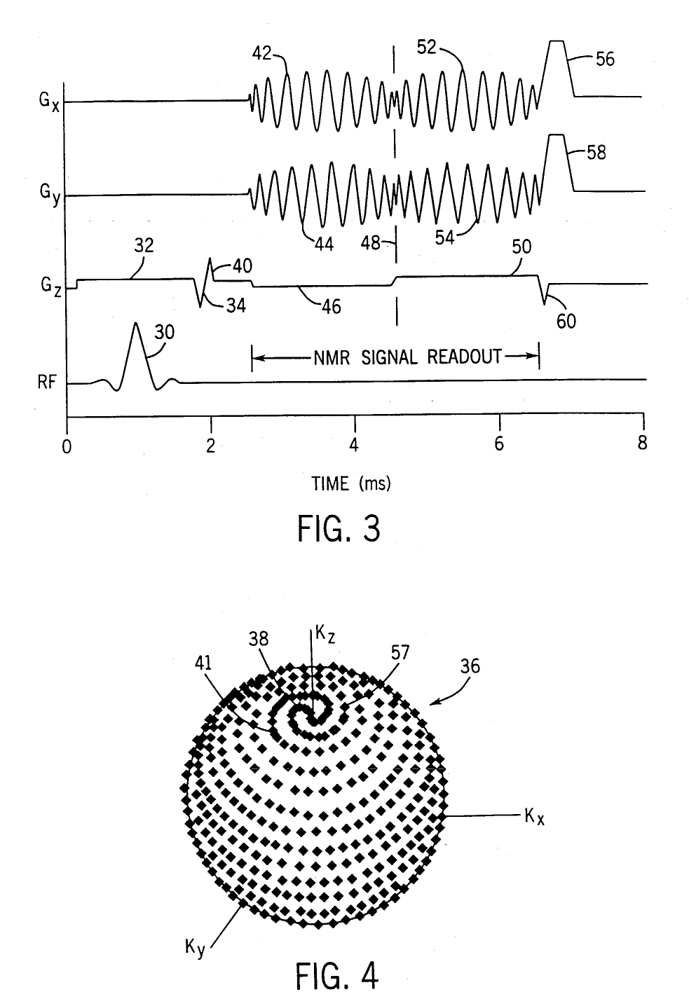

Before ending the MR examination and while the patient is still in the prescribed location, a reference navigator signal (SNAV₀) is acquired as indicated at process block 172. The pulse sequence for doing this is described in detail below with reference to FIG. 3. This step acquires information with virtually no additional scan time (e.g. 16 seconds) that enables the patient's position to be locked in with respect to the MRI system's coordinate system, and with respect to all the individual image slices or volumes in the MRI examination. As indicated at process block 174, the reference navigator signal (SNAV₀) is stored in memory along with all the images acquired during the initial examination and the examination is terminated at process block 176 by removing the patient from the bore of the MRI system magnet.

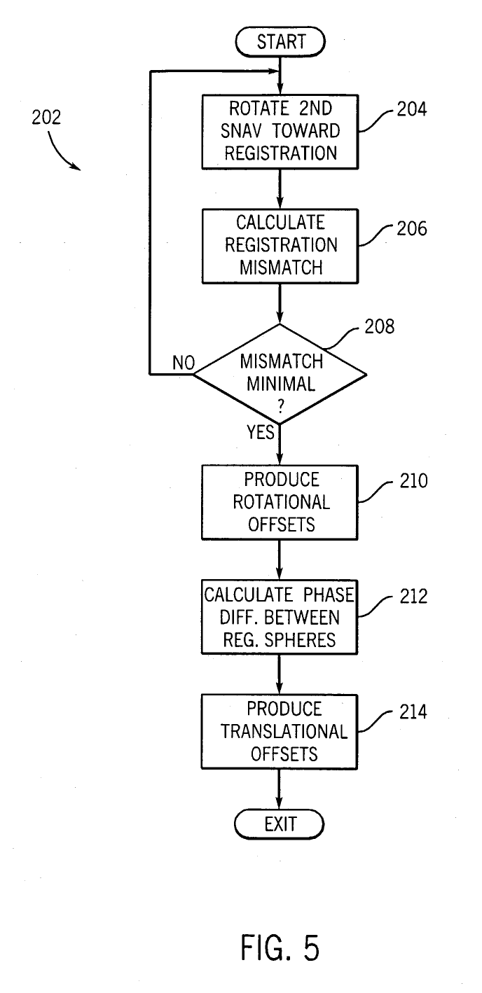

Referring still to FIG. 2, when the patient is subsequently examined, the patient is positioned in the MRI system as indicated at process block 178. Positioning and alignment devices may be used to place the patient in the same location and orientation as the reference examination, but precision is not required. This is followed by acquiring a navigator signal (SNAVₙ) using the pulse sequence of FIG. 3 as indicated at process block 180. As indicated at process block 182, the SNAVₙ signal is employed with the previously acquired and stored reference navigator signal SNAV₀ to calculate the rotational and translational offsets necessary to align the patient exactly with the previous scan. The calculation of these offsets is described in detail below with reference to the flow chart in FIG. 5. These offsets are used to alter the imaging gradient waveforms produced by the imaging pulse sequence during the subsequent prescribed acquisition of MR images. These alterations to the pulse sequence effectively rotate and/or translate, the imaging coordinate system of the MRI system such that the subsequently acquired images all appear in the same orientation and location in the reconstructed images as if the patient were not moved. The calculated offset angle is input as offsetting angles to the oblique imaging feature which is standard on nearly all commercial MRI systems. Similarly, the calculated translational offset along each imaging axis is input to offset the prescribed region of interest by corresponding amounts. As indicated at process block 184, the subsequent images are then acquired.

The reconstructed and aligned subsequent images may then be compared with the reference MR images as indicated at process block 186. The examination is terminated as indicated by process block 188 and the patient is removed from the MRI system. Additional examinations may be performed using the same procedure and all subsequently acquired images are aligned with the reference MR images. As a result, subsequently acquired MR images are also aligned with each other.

There are a number of alternative embodiments of this examination procedure. The navigator signals SNAVₙ acquired with subsequent examinations may be stored along with their associated subsequent MR images. This enables images from two subsequent examinations to be aligned with each other directly, rather than indirectly through the reference navigator signal SNAV₀.

Also, rather than prospectively aligning the acquired images by adjusting scan parameters to offset patient misalignment, the subsequently acquired images can be retrospectively aligned after their acquisition. In this embodiment of the invention the subsequently acquired images are acquired and then an associated navigator signal (SNAVₙ) is acquired using the pulse sequence of FIG. 3. The two navigator signals SNAV₀ and SNAVₙ are employed to align the subsequently acquired images with the stored reference MR images. This is achieved by calculating rotational and translational offsets as described below with reference to FIG. 5 and moving the subsequently acquired images accordingly.

Referring particularly to FIGS. 3 and 4, the spherical navigator pulse sequence includes a volume selective 30° RF excitation pulse 30 which is produced in the presence of a small G_{z} slab select gradient pulse 32 to produce transverse magnetization throughout the region being imaged. For example, if ten slices are acquired during the MR examination, the excited slab includes all ten slices. This is followed by a G_{z} rephasing pulse 34 which has one-half of the area of G_{z} slab select gradient pulse 32. the three gradient fields Gₓ, G_{y} and G_{z} are then manipulated during signal readout to sample three-dimensional k-space on the surface of a sphere 36 centered at the origin of k-space and having a radius K_{ρ}=9.5.

In the preferred embodiment the spherical surface 36 is sampled by a spiral trajectory which starts at a point 38 where k_{z}=k_{ρ}, spirals down to the opposite side, or pole, of the sphere where k_{z}=−k_{ρ}, and then spirals back to the starting point 38. The starting point is established by a G_{z} dephasing gradient pulse 40, and the downward spiral sampling trajectory 41 is produced by sinusoidal G_{z} and G_{y} readout gradients in the presence of a small amplitude, negative G_{z} gradient 46. The spiral sampling trajectory reverses direction at the time indicated by dashed line 48 and the G_{z} gradient switches to a positive value 50. The Gₓ and G_{y} readout gradients 52 and 54 vary sinusoidally to produce a spiral sampling pattern 57 back to the starting point 38. The two spiral sampling patterns 41 and 57 are interleaved such that the surface of the sphere 36 is sampled substantially uniformly throughout. A total of 1952 samples of the NMR navigator signals are acquired during the signal readout. The equations for the three readout gradients during the readout period are as follows:

Physical Parameters

| Symbol | Description | Value |
|---|---|---|
| γ/2π | gyromagneitc ratio | 4257 [Hz/Gauss] |
| Δt | gradient time step | 4e−6 [sec] |
| M | time samples between k-space positions | 2 |
| N | number of k-space samples | 1008 |
| kₚ | k-space radius | 0.396 [cm⁻¹] |
| S_{MAX} | max slew rate | 12,000 [Gauss/cm/sec] |
| G_{MAX} | max gradient strength | 4 [Gauss/cm] |

PHYSICAL EQUATIONS
Given a k-space trajectory: k(t)

| | | |
|---|---|---|
| Gradient Waveforms | $\vec{G} ( t ) = \frac{2 π}{γ} \frac{ⅆ}{ⅆ t} \vec{k} ( t )$ | (1) |
| Slew Rate | $\vec{S} ( t ) = \frac{ⅆ}{ⅆ t} \vec{G} ( t )$ | (2) |
| Continuous Time for Gradient | t = nM Δt = 2n Δt | (3) |

Pole-to-Pole Trajectory (T is Number of turns around the sphere)

| | | | |
|---|---|---|---|
| Latitude | φ(n) | $\frac{πn}{N}$ | (4) |
| Longitude | θ(n) | $\frac{2 πnT}{N}$ | (5) |
| k-space | k_{z} | k_{ρ}cosφ | (6) |
| trajectory | kₓ | k_{ρ}sinφcosθ | (7) |
| | k_{y} | k_{ρ}sinφsinθ | (8) |
| Gradient waveforms | G_{z} | $\frac{2 π}{γ} \frac{ⅆ}{ⅆ t} cos ( \frac{πt}{2 Δt \cdot N} )$ | (9) |
| | Gₓ | $\frac{2 π}{γ} \frac{ⅆ}{ⅆ t} sin ( \frac{πt}{2 Δt \cdot N} ) cos ( \frac{2 πtT}{2 Δt \cdot N} )$ | (10) |
| | G_{y} | $\frac{2 π}{γ} \frac{ⅆ}{ⅆ t} sin ( \frac{πt}{2 Δt \cdot N} ) sin ( \frac{2 πtT}{2 Δt \cdot N} )$ | (11) |

Equator-to-Pole Trajectory

| | | | |
|---|---|---|---|
| k-space trajectory | k_{z}(n) | $\frac{2 n - N - 1}{N}$ | (12) |
| | kₓ(n) | $cos ( \sqrt{N π} {sin}^{- 1} {k}_{z} ( n ) ) \sqrt{1 - {k}_{z}^{2} ( n )}$ | (13) |
| | k_{y}(n) | $sin ( \sqrt{N π} {sin}^{- 1} {k}_{z} ( n ) ) \sqrt{1 - {k}_{z}^{2} ( n )}$ | (14) |
| Gradient waveforms | G_{z}(n) | $\frac{2 π}{γ} \frac{ⅆ}{ⅆ t} ( \frac{\frac{t}{Δt} - N - 1}{N} )$ | (15) |
| | Gₓ(n) | $\frac{2 π}{γ} \frac{ⅆ}{ⅆ t} ( cos ( \sqrt{N π} {sin}^{- 1} ( \frac{\frac{t}{Δt} - N - 1}{N} ) ) \sqrt{1 - {( \frac{\frac{t}{Δt} - N - 1}{N} )}^{2}} )$ | (16) |
| | G_{y}(n) | $\frac{2 π}{γ} \frac{ⅆ}{ⅆ t} ( sin ( \sqrt{N π} {sin}^{- 1} ( \frac{\frac{t}{Δt} - N - 1}{N} ) ) \sqrt{1 - {( \frac{\frac{t}{Δt} - N - 1}{N} )}^{2}} )$ | (17) |

Notice that the trajectory in k_{z}, i.e. from north pole to south pole is not linear. B₀ field inhomogeneity in the physical z direction will produce an apparent (false) z translation. Our solution to this problem is to describe a north-to-south-to-north pole or V-shaped k_{z} trajectory rather than a linear pole-to-pole kz trajectory, so that a phase role in k_{z} due to B₀ inhomogeneities can be distinguished from actual physical translation of the object in k_{z}.

After the navigator signal readout is complete Gₓ and G_{y} spoiler gradient pulses 56 and 58 are applied. A negative G_{z} rewinder gradient pulse 60 is also applied.

While it is preferred to sample the entire surface of k-space sphere 36, good results have also been obtained by sampling less than the entire surface. More specifically, the ability of the MRI system gradient amplifiers 127 to slew the Gₓ and G_{y} readout gradient at a sufficiently high rate to produce the above-described spiral trajectory pattern may limit the ability to sample near the “poles” of the sphere 36 where the sampling pattern spirals more quickly. It has been discovered that up to 15% of the surface can be unsampled without significantly affecting the motion measuring accuracy of the acquired NMR navigator signal. In this case it may be advantageous to sample the spherical surface 36 in two separate excitations. During the first excitation the upper half of the spherical surface 36 is sampled by starting at the “equator” (i.e. k_{z}=0) and spiraling upward toward the north pole (i.e. k_{z}=+k_{ρ}) until the maximum slew rate of the gradient system is reached. This is followed by a second excitation in which the lower half of the spherical surface 36 is sampled by spirally downward from the equator toward the south pole (k_{z}=−k_{ρ}). A total of 1008 samples are acquired during each of these two readouts in this alternative embodiment of the invention.

The processing of the SNAV signals may either be done in real time if prospective alignment is to be performed, or it may be done after the scan is complete if retrospective image alignment is to be performed.

Referring particularly to FIG. 5, the processing of the two navigator signals SNAV₀ and SNAVₙ to align acquired MR images will now be described in detail. As described above with reference to FIG. 3, this procedure is employed to produce an aligned MR image in a subsequent examination by rotating and translating the gradient coordinate system (prospective) or the subsequently acquired image (retrospective) by offset amounts. A loop is entered at 202 in which the acquired spherical navigator k-space data SNAVₙ is rotated until it reaches optimum registration with the reference spherical navigator SNAV₀. This is illustrated in the texture maps of FIGS. 7b and 7c which illustrate by their shading the magnitude values on the k-space sphere. We perform this registration by minimizing a cost function that measures the degree of mismatch between the reference spherical navigator (SNAV₀) data set and the acquired (SNAVₙ) data set as trial rotations are applied to the latter. Initial experiments were performed using the sum squared difference as the cost function and downhill simplex minimization as the optimization algorithm as described by Press et al. “Numerical Recipes in C,” 2^{nd} ed. New York, N.Y.: Cambridge University Press (1992). All three rotation angles (θₓ, θ_{y}, θ_{z}) are solved for simultaneously by the algorithm, which typically requires 20-50 iterations to converge.

Each trial rotation of SNAVₙ is performed at process block 204 and the mismatch between it and the reference navigator signal SNAV₀ is calculated at process block 206. For each sample point of SNAVₙ, its θ and φ coordinates after the rotation are calculated. The corresponding magnitude value for SNAV₀ is calculated using bilinear interpolation of the four sample points that surround these coordinates in θ and φ. The squared difference between the interpolated magnitude value from the reference data SNAV₀ and the measured value from the acquired/rotated data is calculated. The sum of these squared differences for all the sample points on the k-space sphere is the cost function value for the iteration.

When the mismatch is minimized, as indicated at decision block 208, the registration is complete. Otherwise, the system loops back to try another set of rotation angles. As indicated at process block 210 the correction angles which register the two spherical data sets SNAV₀ and SNAVₙ are then produced and used as offsets as described above. This corrects the image data for patient rotational misalignment about any axis in space.

The next step as indicated by process block 212 is to calculate the phase difference at each sample point in the two registered k-space spheres. Translational motion does not alter magnitude values on the spherical shell, but does alter phase values. At each point in 3-D k-space, a translation of (Δₓ, Δ_{y}, Δ_{z}) causes a phase change Δφ according to equation (18). If the spherical shell is sampled with N points, then each point yields an equation of this form, building a system of N equations in 3 unknowns. The calculation

Δφ=2_{Π}[Δxkₓ+Δyk_{y}+Δzk_{z}] (18)

of translation is thus highly over determined and is quite robust. Note that the general process of determining translations after a rotation requires regridding of the points from the original to the rotated grid, which involves the calculation of phase values from interpolated data. Once the phase values are registered by any necessary rotation, the unwrapped phase differences can be plugged into a weighted least squares inversion to find the (Δₓ, Δ_{y}, Δ_{z}) translation. Equations (19-22) below describe the weighted least squares inversion calculation. The 3×1 column vector x contains the unknown motions (Δₓ, Δ_{y}, Δ_{z}). The elements of the N×1 column vector b are the unwrapped phase differences. The rows of the N×3 matrix A contain the (kx, ky, kz) position of each sampled point in k-space. The N×N weighting matrix W has been added in equation (20) to account for higher noise in the phase at low magnitude positions in k-space. After calculating the inverse of the 3×3 matrix Q defined in equation (21), one can find the best least squares fit (Δₓ, Δ_{y}, Δ_{z}) translations in x using equation (22).

Ax=b (19)

(A^{T}WA)x=A^{T}Wb (20)

Q=A^{T}WA (21)

x=Q⁻¹A^{T}Wb (22)

As indicated at process block 214, these translational corrections are produced and used as offsets as described above.

While the use of a spherical navigator signal is preferred because it contains information sufficient to align images for rotational and translational misalignment along all three spatial axes, other navigator signals may also be used. For example, orbital navigator signals as described in the above-cited U.S. Pat. No. 5,539,312 may be employed where subject misalignment is limited to rotation and translation in a single two-dimensional plane.

In the preferred embodiment a single, high resolution spherical NMR navigator signal is acquired during each subsequent examination and the information therein is employed either prospectively or retrospectively to align the images. When a prospective alignment strategy is employed, however, it is also possible to iteratively acquire a plurality of NMR navigator signals and adjust the imaging pulse sequence after each SNAV signal acquisition until the misalignment drops below a preset level. Only then is the subsequent image acquired. This enables a low resolution navigator signal to be used during initial iterations to offset gross misalignment and then a higher resolution navigator signal to be used to provide precise subject alignment.

1. A method for acquiring images during a succession of magnetic resonance examinations of a subject, the steps comprising:
a) positioning the subject in a magnetic resonance imaging (MRI) system;
b) acquiring a prescribed image of the subject by performing an imaging pulse sequence;
c) acquiring associated NMR navigator signal data by performing a navigator signal pulse sequence;
d) storing the prescribed image and associated NMR navigator signal data;
e) removing the subject from the MRI system;
f) re-examining the subject to acquire a subsequent image by repeating steps a), b) and c) and aligning the subject depicted in the prescribed image and the subject depicted in the subsequent image using information in their associated NMR navigator signal data.

2. The method as recited in claim 1 in which the aligning is performed by:
i) analyzing the associated NMR navigator signal data to calculate the rotational misalignment of the subject;
ii) rotating the subsequent image to offset the calculated rotational misalignment;
iii) analyzing the associated NMR navigator signal data to calculate the translational misalignment of the subject; and
iv) translating the subsequent image to offset the calculated translational misalignment.

3. The method as recited in claim 2 in which the subsequent image is comprised of a k-space data set, step ii) is performed by rotating the k-space data with respect to a k-space coordinate system, and step iv) is performed by shifting the phase of the k-space data.

4. The method as recited in claim 1 in which the navigator signal pulse sequence samples the surface of a sphere in k-space and the information in the associated NMR navigator signal data enables alignment around any axis of subject rotation and along any axis of subject translation.

5. The method as recited in claim 1 in which the aligning is performed by:
i) analyzing the associated NMR navigator signal data to calculate the rotational misalignment of the subject
ii) analyzing the associated NMR navigator signal data to calculate the translational misalignment of the subject; and
iii) modifying the imaging pulse sequence used to acquire the subsequent image to offset the calculated rotational and translational misalignment of the subject.

6. The method as recited in claim 5 in which the navigator signal pulse sequence samples the surface of a sphere in k-space and the information in the associated NMR navigator signal data enables alignment around any axis of subject rotation and along any axis of subject translation.

7. A method for performing a series of magnetic resonance imaging examinations of a subject, the steps comprising:
a) positioning the subject in a magnetic resonance imaging (MRI) system;
b) acquiring a prescribed image of the subject by performing an imaging pulse sequence;
c) acquiring associated NMR navigator signal data by performing a spherical navigator signal pulse sequence;
d) storing the prescribed image and associated NMR navigator signal data;
e) re-examining the subject to acquire a subsequent image by repeating steps a), b) and c) and wherein the subject depicted in the prescribed image is aligned with the subject depicted in the subsequent image by translating and rotating the subsequent image using information in their associated NMR navigator signal data.

8. The method as recited in claim 7 in which step e) includes:
i) analyzing the associated NMR navigator signal data to calculate the rotational misalignment of the subject;
ii) rotating the subsequent image to offset the calculated rotational misalignment;
iii) analyzing the associated NMR navigator signal data to calculate the translational misalignment of the subject; and
iv) translating the subsequent image to offset the calculated translational misalignment.

9. The method as recited in claim 8 in which the subsequent image is comprised of a k-space data set, step ii) is performed by rotating the k-space data with respect to a k-space coordinate system, step iv) is performed by shifting the phase of the k-space data, and an aligned image is reconstructed from the rotated and phase shifted k-space data.

10. The method as recited in claim 7 in which the spherical navigator signal pulse sequence samples the surface of a sphere in k-space and the information in the associated NMR navigator signal data enables alignment around any axis of subject rotation and along any axis of subject translation.

11. The method as recited in claim 7 in which step e) includes:
i) analyzing the associated NMR. navigator signal data to calculate the rotational misalignment of the subject
ii) analyzing the associated NMR navigator signal data to calculate the translational misalignment of the subject; and
iii) modifying the imaging pulse sequence used to acquire the subsequent image to offset the calculated rotational and translational misalignment of the subject.

12. The method as recited in claim 11 in which the spherical navigator signal pulse sequence samples the surface of a sphere in k-space and the information in the associated NMR navigator signal data enables alignment around any axis of subject rotation and along any axis of subject translation.
